(19) United States
(12) Patent Application Publication
(10) Pub. No.: US 2002/0156321 A1
(43) Pub. Date: Oct. 24, 2002

(54) Continuous preparation of high purity Bis(fluoroxy)difluoromethane (BDM) at elevated pressure

(76) Inventor: Robert George Syvret, Allentown, PA (US)

Correspondence Address: AIR PRODUCTS AND CHEMICALS, INC., PATENT DEPARTMENT, 7201 HAMILTON BOULEVARD, ALLENTOWN, PA, 181951501

(21) Appl. No.: 09/782,268

(22) Filed: Feb. 13, 2001

(51) Int. Cl. C07C 51/60

(52) U.S. Cl. 562/849

(57) ABSTRACT

The invention provides an improved process for preparing bis(fluoroxy)difluoro-methane (BDM) by continuously reacting F₂ with CO₂ in a reactor containing a fluorination catalyst (e.g., CsF), wherein the process is conducted at a pressure above atmospheric pressure. The process provides BDM of very high purity and very low residual F₂.

### BACKGROUND OF THE INVENTION

This invention relates to a continuous process for preparing bis(fluoroxy)-difluoromethane (BDM) in high yields.

There is a need in the art for scaleable processes which are capable of generating electrophilic fluorination agents (hereinafter referred to as “F⁺” agents) with sufficient “F⁺” character, or alternatively, “F⁺” power, to effect electrophilic fluorination reactions of a wide variety of organic substrates with high selectivity, but yet are safe to produce and use as intended.

In order to achieve high selectivity in electrophilic fluorination reactions, and hence commercial utility, the desired “F⁺” agent must be very low or nearly completely devoid of radical fluorine sources, F^{·}, including F₂. Thus, the most desirable process must be highly efficient in converting F₂ to the “F⁺” agent. An example of a “F⁺” agent which possesses sufficient fluorination (“F⁺”) power and selectivity in fluorination reactions with organic substrates is bis(fluoroxy)difluoromethane, hereinafter referred to as BDM.

BDM can be synthesized by a variety of processes. Hohorst et al., “Bis(fluoroxy)difluoromethane, CF₂(OF)₂.” Journal of the American Chemical Society (1967), Vol.89, pages 1809-1810, prepared BDM in 99.7% yield through the static room temperature reaction between CO₂ and a 305% molar excess of F₂ in the presence of a large molar excess of CsF. Cauble et al. “Preparation of Bis(fluoroxy)difluoromethane, CF₂(OF)₂.” Journal of the American Chemical Society (1967), Vol. 89, page 1962, prepared BDM at room temperature in 99.1% yield using a similar procedure and nearly 100% excess F₂. Cauble et al. “Fluorocarbonyl Hypofluorite.” Journal of the American Chemical Society (1967), Vol. 89, pages 5161-5162, prepared BDM by the reaction of fluorocarbonyl hypofluorite with excess F₂ in the presence of CsF. Lustig et al. “The Catalytic Addition of Fluorine to a Carbonyl Group. Preparation of Fluoroxy Compounds.” Journal of the American Chemical Society (1967), Vol. 89, pages 2841-2843, prepared BDM in 98.0% yield using a similar procedure with 15.7% excess F₂. Thompson et al. Thompson, “Preparation and Characterization of Bis(fluoroxy)perfluoroalkanes. II. Bis(fluoroxy)perfluoromethane.” Journal of the American Chemical Society (1967), Vol. 89, pages 1811-1813 and U.S. Pat. No. 3,420,866 (Prager et al., 1969), disclosed the preparation of BDM by treating sodium trifluoroacetate, perfluorosuccinic anhydride, or sodium oxalate with diluted F₂ (≦50% v/v) in a flow system, and subsequently trapping all volatile products at −186° C.; however, the yields were poor (e.g., 2% yield when using sodium trifluoroacetate and 1-15% when using sodium oxalate). Mulholland et al., “Facile, Temperature-Dependent Formation of C₁ and C₂ Perfluoroalkyl Hypofluorites. Applications as Electrophilic Fluorinating Agents.” Journal of Organic Chemistry (1986), Vol. 51, page 1482, report that BDM was obtained in 5% yield by flowing 10% F₂/N₂ (v/v) through sodium trifluoroacetate, followed by trapping all volatiles at −196° C. U.S. Pat. No. 3,394,163 (Kroon, 1968) discloses the production of BDM by fluorination of alkali metal oxalates followed by trapping the products at −158° C. and subsequent purification of BDM product by distillation. U.S. Pat No. 4,499,024 (Fifolt, 1985) and Michael J. Fifolt et al., “Fluorination of Aromatic Derivatives with Fluoroxytrifluoro-methane and Bis(fluoroxy)difluoromethane.” Journal of Organic Chemistry (1985), Vol. 50, pages 4576-4582 disclose that BDM can be produced in a sequence of steps involving flowing mixtures of F₂ and CO₂ through a bed of activated CsF, collecting the exit gases, and recovering BDM from the collected gases.

Following are some of the shortcomings found in known processes for producing BDM, such as those discussed here:

batch (static) processes require subsequent separation of the BDM product from excess F₂;

BDM-containing product gases, produced in a flow system, must be collected (trapped) and separated from residual F₂; and

when BDM is produced in flow systems, the substrate beds are sacrificial and must be replenished, and the BDM product must be subsequently recovered and purified.

In addition, the above-cited processes which collect BDM product in a low-temperature trap in order to separate it from residual F₂ are quite disadvantageous since: (i) the excess F₂, which is difficult and expensive to make and handle, is wasted, and moreover, must be disposed of (which is neither trivial nor inexpensive); and (ii) condensation of BDM product in a low-temperature trap is potentially very dangerous due to (a) the uncertainty of co-condensing highly energetic and unstable byproducts of the BDM synthesis reaction, and (b) the potential for spark-initiated explosive decomposition of BDM in the condensed phase (discovered by the inventor).

Accordingly, it is desired to provide a safe and commercially scaleable process to prepare BDM, which is low in radical fluorine sources or impurities, including F₂.

### BRIEF SUMMARY OF THE INVENTION

The invention provides an improved process for preparing BDM by continuously reacting F₂ with CO₂ at moderate temperature in a reactor containing a fluorination catalyst, wherein the process is conducted at a pressure above atmospheric pressure. The process provides BDM of very high purity and very low residual F₂, such that the continuous product mixture is useable for fluorination or other applications without collection, isolation, purification or dilution.

### BRIEF DESCRIPTION OF THE DRAWING

FIG. 1 shows an embodiment of a BDM generation system of the invention.

### DETAILED DESCRIPTION OF THE INVENTION

In the present invention, BDM (i.e., CF₂(OF)₂) is produced in a flow system from the reaction between F₂ and CO₂ in the presence of a fluorination catalyst (e.g., CsF) as depicted in the following equation:

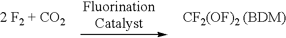

Preferred embodiments of the present invention provide a continuous (flow) process for synthesizing an electrophilic fluorination agent, BDM, with very high purity and very low residual F₂. BDM yields are preferably at least 90%, more preferably at least 95%, most preferably at least 99% (calculated as (experimental/theoretical)×100).

The F₂ utilization efficiency of the present process is also excellent, and is significantly greater than that known in the background art cited above. Thus, product mixtures of the invention preferably contain residual F₂ in amounts less than 1%, more preferably less than 0.6%, most preferably undetectable amounts of F₂ (as determined by on-line UV analysis @311 nm).

Moreover, there is a greater degree of safety inherent in the present invention as compared to the prior art, since in the present case, isolation and purification of the BDM product mixture are not required. Condensation of fluoroxy compounds, including BDM, is quite hazardous since these compounds have the potential to explode violently in concentrated or condensed forms. In fact, the inventor and colleagues have demonstrated that concentrated BDM mixtures will decompose explosively if a spark is introduced to the mixture.

The reaction of the invention is conducted at super-ambient pressure. Preferably, the reaction pressure is at least about 6 psig (142.7 kPa), more preferably at least 75 psig (618.5 kPa), and even more preferably 75 psig to 320 psig (618.5 kPa to 2307.8 kPa).

The reaction is preferably conducted at super-ambient temperature, but can also be conducted at ambient or sub-ambient temperature. Preferably, the temperature is about 5° C. to about 51° C.; more preferably, 20° C. to 38° C.

In preferred embodiments, super-ambient reaction pressures and moderate reaction temperatures of about 20 to 38° C. are used to ensure complete conversion of F₂ and to control or avoid the formation of unwanted byproducts. The resulting product stream is completely, or nearly completely free of residual F₂ and unwanted byproducts and as such, can be used directly for useful purposes, e.g., electrophilic fluorination, oxidation, initiation of radical polymerization processes, and etching, without the need to collect the BDM product and separate it from residual F₂ and unwanted byproducts.

The molar ratio of carbon dioxide to fluorine can range from 0.5 to 25 Typically it ranges from 2.5 to 10.

The residence time on the fluorination catalyst is preferably greater than 0.25 minutes, more preferably 0.3 minutes to 1.3 minutes.

When CsF is used as the catalyst, it should be rigorously dry and totally free of H₂O and HF. The inventor has found that even minimum exposure of the bulk CsF catalyst bed to either H₂O or HF results in immediate and non-reversible poisoning of the catalyst bed to the point of not functioning at all as a catalyst. Thus, the catalyst should preferably be activated (i.e., rendered dry and free of H₂O and HF) prior to use as suggested in the examples, below, or by any other method suitable for the purpose.

Although CsF has been the typical catalyst used in the process to produce BDM from CO₂ and F₂, other known fluorination catalysts can be used in this invention.

The amount of catalyst that is required depends on the intended flow rate of reactant gases, the catalyst bed pressure used, the ratio of CO₂, F₂, and other process parameters.

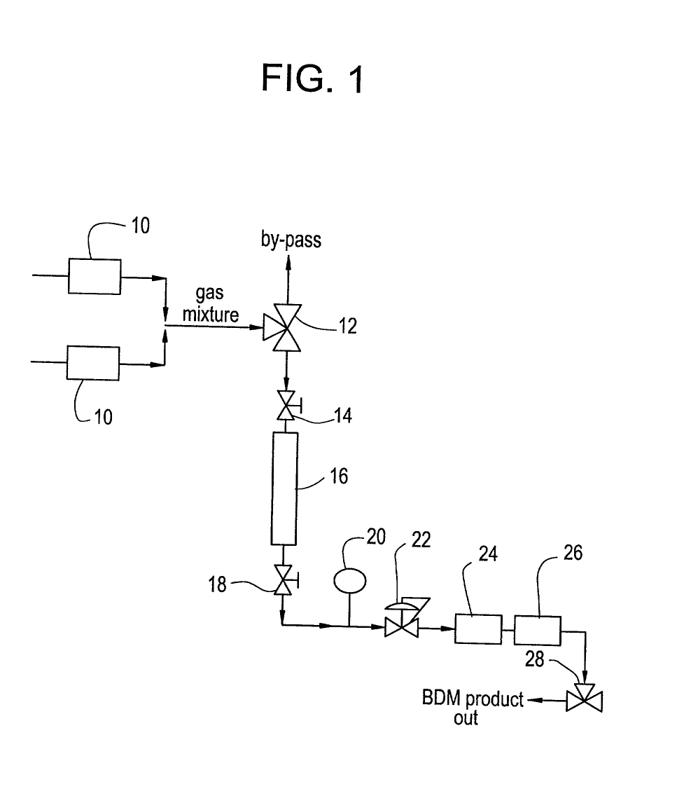

Referring to FIG. 1, the gas streams are mixed downstream of mass flow controllers 10 and passed through a three-way valve 12 and an on/off valve 14 before entering a catalyst bed 16. The reaction product mixture exits bed 16 and passes through an on/off valve 18. The pressure of the flow stream is measured by a pressure indicator 20 downstream of on/off valve 18 and upstream of a back-pressure regulator 22. The pressure in bed 16 is maintained at the preferred super-ambient operating pressure by means of back pressure regulator 22. Upon exiting back pressure regulator 22, the product mixture is passed through an infrared spectrometer 24 for the purpose of monitoring product composition, and through an ultraviolet (UV) spectrometer 26 for the purpose of monitoring for residual F₂, before being diverted through a three-way valve 28 to the point of end use. Note there is some uncertainty in measuring residual F₂ at very low levels because of the interference due to a “tailing” BDM UV adsorption in the same region of the spectrum.

The invention will be illustrated in more detail with reference to the following Examples, but it should be understood that the present invention is not deemed to be limited thereto.

### EXAMPLES

In all the examples, the product stream was monitored for F₂ by UV spectroscopy (F₂ absorption maximum is 314 nm, CO₂ absorption maximum is less than 120 nm, and BDM adsorption maximum is about 195 nm) and composition by FT-IR (Fourier-transform infrared spectroscopy).

### Examples 1-5

These examples demonstrate the effect of CsF catalyst activation using CsF, as well as the effect of using super-ambient catalyst-bed operating pressures, on the rate of BDM formation and F₂ conversion. The CsF catalyst bed temperature for each of Examples 1-5 was 21-23° C. Data are summarized in Table 1. These examples also demonstrate that the BDM product stream can be used directly for selective electrophilic fluorination of an aromatic substrate.

TABLE 1

| | | CO₂ | Total | Reactor | F₂ |
|---|---|---|---|---|---|
| | F₂ Flow | Flow | Flow | pressure | Breakthrough |
| Example | (sccm^{a}) | (sccm) | (sccm) | (psig) | (approx. %)^{b} |
| 1* | 5 | 100 | 125 | 0 | 4 |
| 2a* | 5 | 100 | 125 | 0 | 4 |
| 2b | 5 | 100 | 125 | 36 | 1.9 |
| 2c | 5 | 100 | 125 | 67 | 1.4 |
| 3a | 5 | 100 | 125 | 64 | 0.97 |
| 3b | 5 | 100 | 125 | 73 | 0.90 |
| 4a | 2 | 50 | 60 | 78 | 0.31 |
| 4b | 2 | 50 | 60 | 110 | 0.26 |
| 4c | 2 | 50 | 60 | 120 | 0.15 |
| 5 | 2 | 50 | 60 | 80 | 0.29 |

^{a}Standard cubic centimeters per minute.
^{b}F₂ breakthrough values have not been corrected for BDM interference which may cause these values to be artificially high.
*Comparative Examples.

### Example 1 (Comparative)

Referring to Table 1, 362.2 g of CsF was used directly (without activation) from the vendor (99.9% CsF obtained from Aldrich Chemical Company). Using no CsF catalyst bed pressure (0 psig), 25 sccm flow of 20% F₂/N₂ (v/v) and 100 sccm CO₂ were passed through the catalyst bed and the resulting product mixture was shown to contain a minimal amount of BDM product and about 4% residual F₂.

### Example 2a (Comparative)

Example 1 was repeated with an activated catalyst bed, which had been activated by heating to 165° C. overnight with flowing dry nitrogen. The resulting product mixture was shown to contain a minimal amount of BDM product and about 4% residual F₂.

### Example 2b

Example 2a was repeated with the bed pressure increased from 0 to 36 psig, which resulted in significantly more BDM and less (1.9%) F₂ observed in the product mixture.

### Example 2c

Example 2a was repeated with the bed pressure increased to 67 psig, which resulted in more BDM and less (about 1.4%) residual F₂ in the product mixture.

### Example 3a

Example 2a was repeated with the bed pressure increased to 64 psig, which resulted in a product mixture containing BDM, CO₂ and residual F₂ (approximately 0.97%).

### Example 3b

Example 3a was repeated with the bed pressure increased to 73 psig, which increased the ratio of BDM to CO₂, relative to that of Example 3a. In addition, less residual F₂ was observed in the product mixture of this example than in Example 3a.

### Example 4a

Example 3b was repeated except using flows of 2 sccm F₂ and 50 sccm CO₂ and with the bed pressure increased to 78 psig, which increased the ratio of BDM to CO₂, relative to that of Example 3b. In addition, less residual F₂ was observed in the product mixture of this example than in Example 3b.

### Example 4b

Example 4a was repeated with the bed pressure increased to 110 psig, to yield a product mixture containing good quality BDM, CO₂, and residual F₂ (approximately 0.26%).

### Example 4c

Example 4b was repeated with the bed pressure increased to 120 psig, to yield a product mixture containing good quality BDM and less residual F₂ (approximately 0.15%) than that observed in Example 4b.

### Example 5

Example 4a was repeated with the bed pressure adjusted to 80 psig. The BDM product stream (containing approximately 0.29% residual F₂) was then used directly for an electrophilic aromatic fluorination reaction. Thus, the BDM product was delivered to a stirred solution at −48° C., which contained an aromatic substrate, 1-(4-chlorophenyl)-3-methyl-4-difluoromethyl-1,2,4-triazolin-5-one (3.0 g, 11.6 mmol) and 2.0 g of nitrobenzene (16.3 mmol) in 220 mL solvent (a 10:1 mixture of CHCl₃/CF₃COOH). After the addition of 20 mol % excess BDM was complete, a sample was taken, neutralized, and examined by GC. The sample showed 99.5% conversion with 90% selectivity to the desired product, 1-(4-chloro-2-fluorophenyl)-3-methyl-4-difluoromethyl-1,2,4-triazolin-5-one.

### Examples 6-33

These examples demonstrate how BDM production and F₂ conversion efficiencies are affected by varying certain reaction parameters, such as catalyst bed temperature, pressure, F₂:CO₂ ratio, and residence time. In each of Examples 6-33, the product stream composition was monitored continuously by on-line UV (at 311 nm) and IR. Note that there is a great amount uncertainty associated with F₂ concentration measurements of ≦0.5% by UV; these measurements can be assumed to be dominated by interference due to BDM adsorption at 311 nm.

A BDM generation system was assembled according to FIG. 1. The CsF catalyst bed consisting of a 316-SS cylinder was loaded with 3325.1 g of CsF powder, which had previously been activated by melting, cooling in a dry inert atmosphere, and grinding to a uniform fine consistency.

### Example 6

In this example 1 slpm CO₂ and 1 slpm 20% F₂/N₂ were flowed through the CsF catalyst bed. The bed pressure and temperature were 51 psig and 21° C., respectively Residual F₂ breakthrough was less than 0.2% (not corrected for BDM adsorption or background).

### Example 7

Up to 7 slpm CO₂ and 7 slpm 20% F₂/N₂ were flowed through the CsF catalyst bed. The bed pressure and temperature were up to 230 psig and 28° C., respectively. Residual F₂ breakthrough was less than 0.9% (not corrected for BDM adsorption or background) even at the highest flow rate.

### Example 8

Up to 5 slpm CO₂ and 5 slpm 20% F₂/N₂ were flowed through the CsF catalyst bed. The bed pressure and temperature were up to 207 psig and 51° C., respectively. Residual F₂ breakthrough was less than 0.6% (not corrected for BDM adsorption or background) even at the highest flow rate.

### Examples 9-33

In Examples 9-33, the effects of varying the reaction parameters: CsF catalyst bed temperature and pressure, CO₂/F₂ mol ratio, and residence time, on BDM production efficiency (calculated as BDM observed versus BDM expected) and residual F₂ breakthrough were assessed. The results are summarized in Table 2.

TABLE 2

| | Bed | Bed | CO₂/F₂ | Res. | | | | 20% F₂ |
|---|---|---|---|---|---|---|---|---|
| | Temp^{a} | Pressure | mol | Time | BDM | F₂ Break- | CO₂ flow | flow |
| Ex. | (° C.) | (psig) | ratio | (min) | efficiency | through^{b} | (slpm^{c}) | (slpm) |
| 9 | 29/20 | 204 | 5.0 | 0.65 | 97.4% | 0.3% | 2.02 | 2.02 |
| 10 | 22/15 | 207 | 5.0 | 0.65 | 97.5% | 0.3% | 2.02 | 2.02 |
| 11 | 24/16 | 208 | 5.0 | 0.65 | 96.1% | 0.4% | 2.02 | 2.02 |
| 12 | 27/19 | 102 | 5.0 | 0.65 | 96.0% | 0.4% | 2.02 | 2.02 |
| 13 | 20/14 | 309 | 5.0 | 0.65 | 95.8% | 0.4% | 2.02 | 2.02 |
| 14 | 23/22 | 205 | 2.5 | 0.65 | 94.8% | 0.2% | 1.02 | 2.02 |
| 15 | 20/16 | 208 | 10.0 | 0.65 | 97.6% | 0.2% | 4.04 | 2.02 |
| 16 | 22/18 | 205 | 5.0 | 1.28 | 96.2% | 0.4% | 1.03 | 1.02 |
| 17 | 20/19 | 205 | 5.0 | 1.28 | 97.4% | 0.3% | 1.02 | 1.02 |
| 18 | 26/19 | 205 | 5.0 | 0.32 | 97.0% | 0.3% | 4.05 | 4.04 |
| 19 | 11/11 | 204 | 5.0 | 0.65 | 96.4% | 0.4% | 2.03 | 2.02 |
| 20 | 11/12 | 102 | 2.5 | 1.28 | 94.7% | 0.7% | 0.52 | 1.02 |
| 21 | 10/9 | 104 | 2.5 | 0.32 | 87.9% | 1.6% | 2.02 | 4.04 |
| 22 | 12/12 | 105 | 9.9 | 1.28 | 93.7% | 0.4% | 2.02 | 1.02 |
| 23 | 9/9 | 104 | 10.0 | 0.32 | 76.8% | 1.6% | 8.07 | 4.04 |
| 24 | 10/12 | 305 | 2.6 | 1.29 | 94.6% | 0.7% | 0.52 | 1.01 |
| 25 | 32/22 | 205 | 5.0 | 0.65 | 97.5% | 0.3% | 2.03 | 2.02 |
| 26 | 35/23 | 103 | 2.5 | 1.29 | 96.0% | 0.5% | 0.51 | 1.01 |
| 27 | 35/25 | 104 | 2.5 | 0.32 | 93.1% | 0.9% | 2.03 | 4.03 |
| 28 | 32/22 | 104 | 10.0 | 1.28 | 99.6% | 0.0% | 2.03 | 1.02 |
| 29 | 35/33 | 104 | 10.0 | 0.33 | 91.9% | 0.5% | 8.02 | 4.02 |
| 30 | 30/29 | 103 | 10.0 | 0.32 | 89.5% | 0.7% | 8.07 | 4.04 |
| 31 | 33/27 | 309 | 2.5 | 1.28 | 83.5% | 2.2% | 0.51 | 1.02 |
| 32 | 36/35 | 207 | 10.0 | 0.32 | >99.9% | 0.0% | 8.07 | 4.04 |
| 33 | 25/23 | 208 | 10.0 | 0.32 | >99.9% | 0.0% | 8.07 | 4.04 |

^{a}The bed temperature is given as upper-zone temperature/lower-zone temperature, wherein the gas flow is from upper-zone to lower-zone.
^{b}BDM efficiency is calculated as observed/theoretical.

The data in Table 2 show that BDM production is efficient at a variety of reaction parameters, as long as the pressure is above atmospheric.

### Example 34

Approximately 360 g of CsF recovered from a previously used catalyst bed was combined with 300 g of fresh CsF from Aldrich and was activated by melting in a Pt crucible, quickly transferring the crucible and melt to the dry atmosphere of a glove box, and following cooling, was ground to a very fine consistency using a grinder. The resulting CsF powder weighing 537 g was transferred under these anhydrous conditions to the BDM reactor and then evaluated for its catalytic activity using a variety of flow and bed pressure settings. The results are summarized in Table 3.

TABLE 3
Fluorination of CO₂ with F₂ Over Activated CsF

| 20% F₂ | CO₂ | Total | Molar | CsF Bed | Vol % BDM | |
|---|---|---|---|---|---|---|
| Flow | Flow | Flow | Ratio | Pressure | Concentration | % F₂ |
| (sccm) | (sccm) | (sccm) | F₂:CO₂ | (psig) | at Outlet | Breakthrough |
| 10 | 50 | 60 | 0.04 | 9 | 1.7 | 0.0 |
| 20 | 50 | 70 | 0.08 | 7 | 3.0 | 0.0 |
| 30 | 50 | 80 | 0.12 | 7 | 4.1 | 00 |
| 40 | 50 | 90 | 0.16 | 7 | 4.9 | 0.0 |
| 50 | 50 | 100 | 0.20 | 8 | 5.6 | 0.0 |
| 60 | 50 | 110 | 0.24 | 6 | 6.1 | 0.0 |
| 70 | 50 | 120 | 0.28 | 6 | 6.6 | 0.0 |
| 80 | 50 | 130 | 0.32 | 6 | 7.0 | 0.0 |
| 90 | 50 | 140 | 0.36 | 6 | 7.4 | 0.0 |
| 100 | 50 | 150 | 0.40 | 6 | 7.7 | 0.0 |

The data in Table 3 show that at pressures as low as 6 psig, there is no fluorine breakthrough, indicating the effectiveness of even a very low pressure for complete conversion of fluorine and carbon dioxide to BDM.

While the invention has been described in detail and with reference to specific examples thereof, it will be apparent to one skilled in the art that various changes and modifications can be made therein without departing from the spirit and scope thereof.

What is claimed is:

1. A continuous process for preparing bis(fluoroxy)difluoromethane (BDM), comprising passing F₂ with CO₂ through a fluorination catalyst, at a moderate temperature and a pressure that is above atmospheric pressure.

2. The process of claim 1, wherein said pressure is at least about 6 psig.

3. The process of claim 1, wherein said pressure is at least 75 psig.

4. The process of claim 1, wherein said pressure is 75 psig to 320 psig.

5. The process of claim 1, wherein a residence time of said CO₂ and said F₂ on said fluorination catalyst is greater than 0.25 minutes.

6. The process of claim 5, wherein said residence time is 0.3 minutes to 1.3 minutes.

7. The process of claim 6, wherein said pressure is at least 75 psig.

8. The process of claim 7, wherein the temperature in said reactor is 20° C. to 38° C.

9. The process of claim 1, wherein said process provides a reaction product gas containing less than 1% F₂.

10. The process of claim 1, wherein a yield of BDM is at least 90%.

11. The process of claim 1, wherein said fluorination catalyst is CsF.
